(19) United States
(12) Patent Application Publication
(10) Pub. No.: US 2017/0181981 A1
(43) Pub. Date: Jun. 29, 2017

(54) BIODEGRADABLE FREE-STANDING CONTROLLED DRUG RELEASE STICKERS

(71) Applicant: Massachusetts Institute of Technology, Cambridge, MA (US)

(72) Inventors: Paula T. Hammond, Newton, MA (US); Bryan Boen Hsu, Thousand Oaks, CA (US); Samantha R. Hagerman, Conshohocken, PA (US)

(21) Appl. No.: 15/392,195

(22) Filed: Dec. 28, 2016

Related U.S. Application Data
(60) Provisional application No. 62/272,433, filed on Dec. 29, 2015.

(51) Int. Cl. A61K 9/70; A61K 38/14; A61K 47/32; A61K 47/34; A61K 47/36

(52) U.S. Cl. CPC A61K 9/7069; A61K 47/34; A61K 47/36; A61K 47/32; A61K 9/7053; A61K 38/14

(57) ABSTRACT

A multi-layer film useful for controlled drug delivery is described. The multilayer film is peelable and self-supporting.

### RELATED APPLICATION

This application claims the benefit of U.S. Provisional Application No. 62/272,433, filed on Dec. 29, 2015, the entire teachings of which are incorporated by reference.

### GOVERNMENT SUPPORT

This invention was made with Government support under Contract No. W911NF-13-D-0001 awarded by the U.S. Army Research Office. The Government has certain rights in the invention.

### BACKGROUND OF THE INVENTION

Judicious placement of controlled drug release systems can improve therapeutic outcomes by producing locally high drug concentrations, thus minimizing the side effects from systemic administration. Although this has led to the promise of personalized therapies with drug loadings and release properties tailored to individual needs, current coatings remain “pre-packaged” and lack customizability. Furthermore, the coating process often requires extensive pre-planning, technical expertise, specialized equipment, and processing conditions that can be potentially drug-inactivating (e.g., high temperatures and harsh solvents).

Also, past free-standing films have required special treatment including covalent crosslinking, non-degradable or inorganic materials, inert surfaces, supportive layers, or substrate dissolution using potentially toxic solutions (e.g., organic solvents or hydrofluoric acid). Taken together, these specialized modifications can make scale-up difficult, result in non-degradable films with poor biocompatibility, or can deleteriously impact therapeutic function through poor drug loading, uncontrolled drug release, or drug inactivation from harsh processing conditions. Furthermore, adhesiveness is often not an inherent property of free-standing films and so a glue needs to be supplemented to bond the coating with the implant.

### SUMMARY OF THE INVENTION

The present disclosure describes fabrication of biodegradable controlled drug release thin films akin to stickers. Using electrostatically assembled layer-by-layer (LbL) films composed of biodegradable polyelectrolytes, it was discovered that tuning the molecular conformation of polymers within the film, as controlled by pH of deposition, could confer peelability to previously non-peelable films, as in Example 1. The films can then be removed from the substrate and stored dry until needed, when the films can be peeled and re-adhered to a new surface within seconds while maintaining controlled drug release properties. These stickers not only retained their controlled release properties upon bonding to new substrates, but were also unaffected by other adjacent or overlapping stickers. FIGS. 6A-6C show that drug release is unaffected when multiple stickers are overlapped.

The present disclosure demonstrates that it is possible to create controlled drug release stickers using the intrinsic intermolecular interactions of the biodegradable polymer/protein components under benign aqueous conditions. It is anticipated that the fabrication of multifunctional coatings independent from the implant will resolve many common manufacturing challenges. Furthermore, the possibility to “mix-and-match” different coatings with implants without affecting the drug loading or release properties would not only empower medical professionals to customize treatment to their patient's specific needs, but also enable them to act intra-operatively with the most recent data available.

In some embodiments, the present disclosure provides a multilayer film comprising repeating multilayer units, wherein each multilayer unit comprises a conformationally flexible polyelectrolyte layer comprising a conformationally flexible polyelectrolyte. In some embodiments, the present disclosure provides a method of assembling a multilayer film, comprising providing a substrate; applying a solution of a polycation, wherein the solution has a pH of less than 5.0 followed by applying a solution of a polyanion; repeating the applying steps a number of times to create a multilayer film; and removing the multilayer film from the substrate.

### BRIEF DESCRIPTION OF THE DRAWINGS

FIGS. 1A-1F are photographs showing steps of a process of peeling of a layer-by-layer (LbL) film from a silicon substrate. In the sequential progression of panels A through F, a (Poly 2/heparin/lysozyme/heparin)₂₄₀ film was peeled from the surface using tweezers within a few seconds.

FIGS. 2A-2G depict properties of various spray-LbL assembled thin films. Panel A is a bar plot illustrating how film thickness, film hardness, and an assessment of “peelability” vary for various films with different numbers of bilayers. Panel B is a schematic representation of multilayer films where the polyelectrolyte layers are in an extended, rigid configuration. Panel C is a schematic representation of multilayer films where the presence of conformationally flexible polyelectrolytes confer peelability. Panel D is a bar plot illustrating how film hardness and bilayer thickness vary for various films with different components and numbers of bilayers. Panel E is a bar plot illustrating how film hardness, bilayer thickness, and an assessment of peelability vary for films with different polyanion pHs. Panel F is a bar plot illustrating how film hardness, bilayer thickness, and peelability vary for films with different hyaluronic acid molecular weights. Panel G is a bar plot illustrating how film tensile strength varies with film composition.

FIGS. 3A-3E are photographs and plots showing properties of drug delivery stickers. Panel A is a photograph showing, in the left portion, a free-standing (Poly 2/heparin/lysozyme/heparin)₂₄₀ film that can be re-adhered to various biomedically important materials, and in the right portion, that film adhered to stainless steel, titanium, and a gelatin sponge. In the right portion of panel A, the arrows indicate the corners of the re-adhered film. Panel B is a graph illustrating the release properties of vancomycin from (Poly 2/heparin/vancomycin/heparin)₂₄₀ films as deposited and as re-adhered to silicon (Si), stainless steel (SS), and titanium (Ti). Panel C is a graph illustrating the release properties of vancomycin from (Poly 2/Alg/vancomycin/heparin)₂₄₀ films as deposited and as re-adhered to silicon (Si). Panel D is a graph illustrating the release properties of vancomycin from (Poly 2/heparin/lysozyme/heparin)₂₄₀ films as deposited and as re-adhered to silicon (Si). Panel E is a photograph showing the results of a Kirby Bauer assay of the antimicrobial activity of (Poly 2/Alg/vanco/Alg)₂₄₀ films against Staphyloccus aureas from (i) as-deposited films on Si, (ii) films re-adhered to Si, (iii) a vancomycin diffusion disc, and (iv) a plain piece of Si.

FIGS. 4A-4F are photographs and schematics showing properties of multi-film sticker composites. Panel A is a photograph showing three multilayer film stickers re-adhered to a silicon wafer on top of each other. Panel B is a schematic showing that the top film includes lysozyme tagged with a red fluorescent label; the middle film includes lysozyme tagged with a green fluorescent label; and the bottom film includes lysozyme tagged with a blue fluorescent label. Panel C is a photograph by a confocal microscope showing how air bubbles can be trapped between layers if desired. Panel D is a photograph by a confocal microscope showing, in a cross-sectional view of the film, that the layers remain distinct. Panel F is a series of photographs by a confocal microscope beginning from the top and progressing into the film showing that each sticker is in close contact with the next.

FIGS. 5A-5D are photographs showing re-adherence of films to various materials. Panel A shows (Poly 2/heparin/vancomycin/heparin)₂₄₀ readhered to silicon. Panel B shows (Poly 2/heparin/vancomycin/heparin)₂₄₀ readhered to stainless steel. Panel C shows (Poly 2/heparin/vancomycin/heparin)₂₄₀ readhered to titanium. Panel D shows (Poly 2/heparin/vancomycin/heparin)₂₄₀ readhered to gelatin sponge.

FIGS. 6A-6C are plots showing release profiles from stacked films. Panel A illustrates release of fluorescently labeled lysozyme from two films peeled and then re-adhered next to each other on a silicon substrate. Panel B illustrates release of fluorescently labeled lysozyme from two films peeled and then re-adhered in a stacked configuration. Panel C illustrates release of fluorescently labeled lysozyme from three films re-adhered in a stacked configuration.

### DEFINITIONS

In order for the present disclosure to be more readily understood, certain terms are first defined below.

As used herein, “or” means “and/or” unless stated otherwise. As used in this application, the term “comprise” and variations of the term, such as “comprising” and “comprises,” have their understood meaning in the art of patent drafting and are inclusive rather than exclusive, for example, of additional additives, components, integers or steps. As used in this application, the terms “about” and “approximately” have their art-understood meanings; use of one vs the other does not necessarily imply different scope. Unless otherwise indicated, numerals used in this application, with or without a modifying term such as “about” or “approximately”, should be understood to cover normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated reference value unless otherwise stated or otherwise evident from the context (except where such number would exceed 100% of a possible value).

As used herein, the term “associated” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated entities are covalently linked to one another. In some embodiments, associated entities are non-covalently linked. In some embodiments, associated entities are linked to one another by specific non-covalent interactions (i.e., by interactions between interacting ligands that discriminate between their interaction partner and other entities present in the context of use, such as, for example. streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

As used herein, the term “biodegradable” is used to refer to materials that, when introduced into cells, are broken down by cellular machinery (e.g., enzymatic degradation) or by hydrolysis into components that cells can either reuse or dispose of without significant toxic effect(s) on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis. In some embodiments, biodegradable polymeric materials break down into their component and/or into fragments thereof (e.g., into monomeric or submonomeric species). In some embodiments, breakdown of biodegradable materials (including, for example, biodegradable polymeric materials) includes hydrolysis of ester bonds. In some embodiments, breakdown of materials (including, for example, biodegradable polymeric materials) includes cleavage of urethane linkages.

The term “conformationally flexible layer” refers to a layer or a film that can be bend or otherwise manipulated without breaking. A conformationally flexible layer will comprise or consist of a conformationally flexible polymer. Without being bound by theory, for example, a conformationally flexible layer may in a rest state consist of conformationally flexible polymers in a contracted configuration. When the conformationally flexible layer is stretched or bent, the contracted polymers are able to reversibly transition into extended configurations as necessary to accommodate the strain being put on the layer, thus allowing the layer as a whole to bend or stretch without breaking. When then bending or stretching force is then removed, the polymers can transition back into the contracted state. The ability of a conformationally flexible polymer to exist in a contracted rest state within a layer will depend on the properties of the polymer and of the environment around it. Without being bound by theory, intra- and inter-molecular interactions of polymers within the layer will affect the propensity of polymers to exist in a contracted configuration. As will be understood by the skilled practitioner, for example, polymers with more favorable inter- and intra-molecular interactions will be more able to rest in a contracted configuration, whereas polymers with less favorable inter- and intra-molecular interactions will be less able to do so. However, polymers with very favorable inter- and intra-molecular interactions may not be able to easily enter an extended conformation. The interactions that affect the rest state of the polymers in a layer include hydrogen bonding, ionic interactions, dipole interactions, Van der Waals forces, hydrophobic packing, and the dielectric shielding provided by the environment.

The term “conformationally flexible,” when referring to a polymer or a molecule, including a macromolecule, refers to an ability of such polymer or a molecule to adopt two or more spatial arrangements of atoms (conformations) and the ability to undergo reversible transitions (conformational changes) between or among such conformations based on the environmental conditions such as acidity, temperature, the presence and nature of a solvent, or external forces. A person of ordinary skill in the art would understand that a polymer or a molecule would fold into one or more specific spatial conformations driven by a number of non-covalent interactions such as hydrogen bonding, ionic interactions, dipole interactions, Van der Waals forces, hydrophobic packing, and the dielectric shielding provided by the environment.

As used herein, the term “hydrolytically degradable” is used to refer to materials that degrade by hydrolytic cleavage. In some embodiments, hydrolytically degradable materials degrade in water. In some embodiments, hydrolytically degradable materials degrade in water in the absence of any other agents or materials. In some embodiments, hydrolytically degradable materials degrade completely by hydrolytic cleavage, e.g., in water. By contrast, the term “non-hydrolytically degradable” typically refers to materials that do not fully degrade by hydrolytic cleavage and/or in the presence of water (e.g., in the sole presence of water).

The term “pH” as used herein, when used to describe a property of a polyelectrolyte layer within a film, refers to the pH of the solution from which that polyelectrolyte layer was deposited.

The phrase “physiological conditions”, as used herein, relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

The term “polyelectrolyte”, as used herein, refers to a polymer which under a particular set of conditions (e.g., physiological conditions) has a net positive or negative charge. In some embodiments, a polyelectrolyte is or comprises a polycation; in some embodiments, a polyelectrolyte is or comprises a polyanion. Polycations have a net positive charge and polyanions have a net negative charge. The net charge of a given polyelectrolyte may depend on the surrounding chemical conditions, e.g., on the pH.

The term “self-supporting film”, as used herein, refers to a film that maintains its structural integrity when not attached to a substrate or other support structure.

As used herein, the term “small molecule” is used to refer to molecules, whether naturally-occurring or artificially created (e.g., via chemical synthesis), that have a relatively low molecular weight. Typically, small molecules are monomeric and have a molecular weight of less than about 1500 g/mol. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§330.5, 331 through 361, and 440 through 460; drugs for veterinary use listed by the FDA under 21 C.F.R. §§500 through 589, incorporated herein by reference, are all considered acceptable for use in accordance with the present application.

As used herein, the term “substantially”, and grammatic equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

The term “therapeutic agent”, as used herein, refers to a substance capable of treating one or more symptoms or features of a particular disease, disorder, and/or condition.

As used herein, the term “treating” refers to partially or completely alleviating, ameliorating, relieving, inhibiting, preventing (for at least a period of time), delaying onset of, reducing severity of, reducing frequency of and/or reducing incidence of one or more symptoms or features of a particular disease, disorder, and/or condition. In some embodiments, treatment may be administered to a subject who does not exhibit symptoms, signs, or characteristics of a disease and/or exhibits only early symptoms, signs, and/or characteristics of the disease, for example for the purpose of decreasing the risk of developing pathology associated with the disease. In some embodiments, treatment may be administered after development of one or more symptoms, signs, and/or characteristics of the disease.

The term “alkyl,” as used herein, refers to a saturated aliphatic branched or straight-chain monovalent hydrocarbon radical having the specified total number of carbon atoms. Thus, “C₁-C₆ alkyl” means a radical having from 1-6 carbon atoms, inclusive of any substituents, in a linear or branched arrangement. Examples of “C₁-C₆ alkyl” include n-propyl, i-propyl, n-butyl, i-butyl, sec-butyl, t-butyl, n-pentyl, n-hexyl, 2-methylbutyl, 2-methylpentyl, 2-ethylbutyl, 3-methylpentyl, and 4-methylpentyl. An alkyl can be optionally substituted with halogen, —OH, C₁-C₆ alkyl, C₂-C₆ alkenyl, C₂-C₆ alkynyl, C₁-C₆ alkoxy, —NO₂, —CN, and —N(R¹)(R²) wherein R¹ and R² are each independently selected from —H and C₁-C₃ alkyl.

The term “alkenyl,” as used herein, refers to a straight-chain or branched alkyl group having one or more carbon-carbon double bonds and having the specified total number of carbon atoms. Thus, “C₂-C₆ alkenyl” means a radical having 2-6 carbon atoms, inclusive of any substituents, in a linear or branched arrangement having one or more double bonds. Examples of “C₂-C₆ alkenyl” include ethenyl, propenyl, butenyl, pentenyl, hexenyl, butadienyl, pentadienyl, and hexadienyl. An alkenyl can be optionally substituted with the substituents listed above with respect to alkyl.

The term “alkynyl,” as used herein, refers to a straight-chain or branched alkyl group having one or more carbon-carbon triple bonds. Thus, “C₂-C₆ alkynyl” means a radical having 2-6 carbon atoms, inclusive of any substituents, in a linear or branched arrangement having one or more triple bonds. Examples of C₂-C₆ “alkynyl” include ethynyl, propynyl, butynyl, pentynyl, and hexynyl. An alkynyl can be optionally substituted with the substituents listed above with respect to alkyl.

The term “cycloalkyl,” as used herein, refers to a saturated monocyclic or fused polycyclic ring system containing from 3-12 carbon ring atoms. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2]bicyclooctane, decahydronaphthalene and adamantane. A cycloalkyl can be optionally substituted with the substituents listed above with respect to alkyl. A “cycloalkenyl” group is a cyclic hydrocarbon containing one or more double bonds. A “cycloalkynyl” group is a cyclic hydrocarbon containing one or more triple bonds.

The terms “heterocyclyl”, “heterocycle”, and “heterocyclic”, as used herein, refer to substituted or unsubstituted non-aromatic ring structures, preferably 3- to 12-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “amino,” as used herein, means an “—NH₂,” an “NHR^{p},” or an “NR^{p}R^{q},” group, wherein R^{p} and R^{q}, each independently, can be C₁-C₁₂ alkyl, C₂-C₁₂ alkenyl, C₂-C₁₂ alkynyl, C₂-C₁₂ alkoxy, cycloalkyl, C₆-C₁₈ aryl, or 5-20 atom heteroaryl. Aminos may be primary (NH₂), secondary (NHRₚ) or tertiary (NRₚR_{q}).

The term “alkylamino,” as used herein, refers to an “NHRₚ,” or an “NRₚR_{q}” group, wherein Rₚ and R_{q} can be alkyl, alkenyl, alkynyl, alkoxy, or cycloalkyl. The term “dialkylamino,” as used herein, refers to an “NRₚR_{q}” group, wherein Rₚ and R_{q} can be alkyl, alkenyl, alkynyl, alkoxy, or cycloalkyl.

The term “alkoxy”, as used herein, refers to an “alkyl-O” group, wherein alkyl is defined above. Examples of alkoxy group include methoxy or ethoxy groups. The “alkyl” portion of alkoxy can be optionally substituted as described above with respect to alkyl.

The term “aryl,” as used herein, refers to an aromatic monocyclic or polycyclic ring system consisting of carbon atoms. Thus, “C₆-C₁₈ aryl” is a monocylic or polycyclic ring system containing from 6 to 18 carbon atoms. Examples of aryl groups include phenyl, indenyl, naphthyl, azulenyl, heptalenyl, biphenyl, indacenyl, acenaphthylenyl, fluorenyl, phenalenyl, phenanthrenyl, anthracenyl, cyclopentacyclooctenyl or benzocyclooctenyl. An aryl can be optionally substituted with halogen, —OH, C₁-C₆ alkyl, C₂-C₆ alkenyl, C₂-C₆ alkynyl, C₁-C₆ haloalkyl, C₁-C₆ alkoxy, C₆-C₁₈ aryl, C₆-C₁₈ haloaryl, (5-20 atom) heteroaryl, —C(O)C₁-C₃ haloalkyl, —S(O)₂—, —NO₂, —CN, and oxo. In an example embodiment, if an aryl is substituted with C₆-C₁₈ aryl, C₆-C₁₈ haloaryl, or (5-20 atom) heteroaryl, those substituents are not themselves substituted with C₆-C₁₈ aryl, C₆-C₁₈ haloaryl, or (5-20 atom) heteroaryl.

The terms “halogen,” or “halo,” as used herein, refer to fluorine, chlorine, bromine, or iodine.

The term “heteroaryl,” as used herein, refers a monocyclic or fused polycyclic aromatic ring containing one or more heteroatoms, such as oxygen, nitrogen, or sulfur. For example, a heteroaryl can be a “5-20 atom heteroaryl,” which means a 5 to 20 membered monocyclic or fused polycyclic aromatic ring containing at least one heteroatom. Examples of heteroaryl groups include pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl. A heteroaryl can be optionally substituted with the same substituents listed above with respect to aryl.

The term “haloalkyl,” as used herein, includes an alkyl substituted with one or more of F, Cl, Br, or I, wherein alkyl is defined above. The “alkyl” portion of haloalkyl can be optionally substituted as described above with respect to alkyl.

The term “haloaryl,” as used herein, includes an aryl substituted with one or more of F, Cl, Br, or I, wherein aryl is defined above. The “aryl” portion of haloaryl can be optionally substituted as described above with respect to aryl.

The term “oxo,” as used herein, refers to ═O.

The term “nitro,” as used herein, refers to —NO₂.

Various types of polymers are defined by the linkages between their repeating units. The term, “polyester”, as used herein, refers to a polymer in which the repeating units are linked by ester groups:

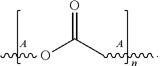

The term “polyanhydride” as used herein, refers to a polymer in which the repeating units are linked by anhydride groups:

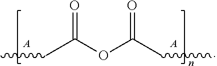

The term, “polyorthoester”, as used herein, refers to a polymer in which the repeating units are linked by orthoester groups. Examples of polyorthoesters include the following:

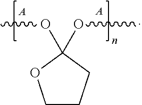

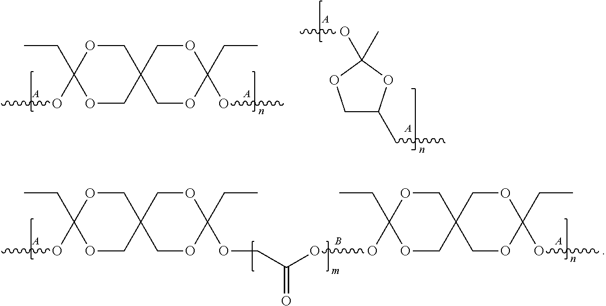

The term, “polyphosphazene”, as used herein, refers to a polymer in which the repeating units are linked by ester groups:

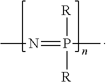

The term, “polyphosphoester”, as used herein, refers to a polymer in which the repeating units are linked by phosphoester groups:

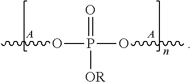

The term, “poly(β-amino ester)”, as used herein, refers to a polyester where the repeating unit contains at least one amino group separated by two carbons from the carboxyl of the ester. Typically, poly(β-amino ester)s have one or more tertiary amines in the backbone of the polymer, preferably one or two per repeating backbone unit. Exemplary poly(β-amino ester)s include the following:

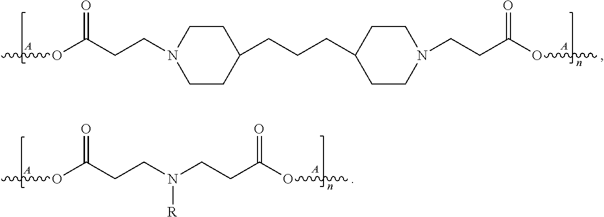

In the above structures, exemplary R groups include hydrogen, branched and unbranched alkyl, branched and unbranched alkenyl, branched and unbranched alkynyl, aryl, halogen, hydroxyl, alkoxy, carbamoyl, carboxyl ester, carbonyldioxyl, amide, thiohydroxyl, alkylthioether, amino, alkylamino, dialkylamino, trialkylamino, cyano, ureido, a substituted acyl group, cycloalkyl, aromatic, heterocyclic, and heteroaryl groups, each of which may be substituted with at least one substituent selected from the group consisting of branched and unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups. Exemplary linker groups A and B, which may be independently selected, include carbon chains of 1 to 30 carbon atoms, heteroatom-containing carbon chains of 1 to 30 atoms, and may be substituted with at least one substituent selected from the group consisting of branched and unbranched alkyl, branched and unbranched alkenyl, branched and unbranched alkynyl, alkylene, alkenylene, alkynylene, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups. The polymer may include, for example, between 5 and 10,000 repeat units. Further examples of poly(β-amino ester)s are poly 1:

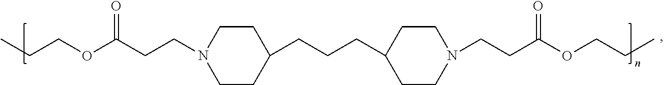

and poly 2:

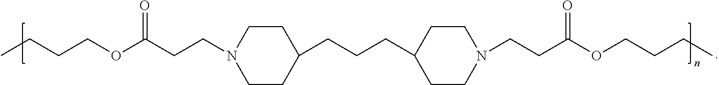

### DETAILED DESCRIPTION OF THE INVENTION

In some embodiments, the present disclosure provides a multilayer film comprising repeating multilayer units, and each multilayer unit comprises a conformationally flexible layer that in turn comprises a conformationally flexible polyelectrolyte. In further embodiments, the conformationally flexible polyelectrolyte is a conformationally flexible polyanion. In further embodiments, each multilayer unit comprises at least one layer containing a polycation. In further embodiments, the multilayer film comprises at least 15, at least 30, or at least 60 multilayer units. In some embodiments, the pH of each conformationally flexibly layers is less than or equal to 5.0. In further embodiments, the pH is less than or equal to 4.5 or less than or equal to 4.0. In further embodiments, the multilayer film is self-supporting. In other embodiments, the polycation is a polyester, a polyanhydride, a polyorthoester, a polyphosphazene, or a polyphosphoester. In further embodiments, the polycation is poly(L-lactide-co-L-lysine), poly(serine ester), poly(4-hydroxy-L-proline ester), or poly[α-(4-aminobutyl)-L-glycolic acid]. In further embodiments, the polycation is a poly(β-amino ester). In further embodiments, the poly(β-amino ester) is a polymer having a repeat unit represented by the following structural formula:

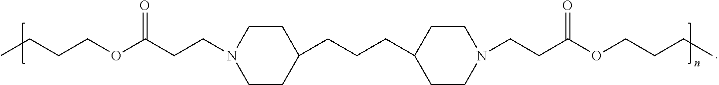

In other embodiments, each conformationally flexible layer includes a polymer selected from sodium polystyrene sulfonate, hyaluronic acid, dextran sulfate, alginate, poly-L-glutamic acid, polyacrylic acid, and chondroitin sulfate. In further embodiments, the conformationally flexible layer includes alginate at a pH of 4.5 or 4.0, or hyaluronic acid at a pH of 4.5. In other embodiments, the repeating multilayer units also include a therapeutic agent, a biomolecule, a small molecule, or a bioactive agent. In other embodiments, the repeating multi-layer units have an average thickness of at least 25 nm, or at least 30 nm.

In some embodiments, the present disclosure provides a system for applying successive layers of materials to a substrate, comprising a source of a cationic solution; a source of an anionic solution; a source of a rinsing fluid; a gas supply connected to the source of the cationic solution, the source of the anionic solution, and the source of the rinsing fluid; a first atomizing nozzle in fluid communication with the source of the cationic solution and adapted to spray the cationic solution toward said substrate; a first atomizing nozzle in fluid communication with the source of the anionic solution and adapted to spray the anionic solution toward said substrate; a first atomizing nozzle in fluid communication with the source of the rinsing fluid and adapted to spray the rinsing fluid toward said substrate; wherein the first atomizing nozzle, the second atomizing nozzle, and the third atomizing nozzle are positioned less than 10 cm from the substrate; and further wherein the anionic solution has a pH of less than 5.0.

In some embodiments, the present disclosure provides a method of assembling a multilayer film, comprising providing a substrate; applying to the substrate a solution of a conformationally flexible polycation with a pH of less than or equal to 5.0, thereby depositing a conformationally flexible layer; and applying a solution of a polyanion to the conformationally flexible layer, thereby depositing a polyanion layer. In a further embodiment, the solution of the conformationally flexible polycation with a pH of less than or equal to 5.0 is applied again, thereby depositing a second conformationally flexible layer; and the solution of the polyanion is applied to the second conformationally flexible layer, thereby depositing at least a second polyanion layer. This results in there being a plurality of multilayer units on the substrate. The plurality of multilayer units is then removed from the substrate.

Any degradable polyelectrolyte can be used in a thin film of the present invention, including, but not limited to, hydrolytically degradable, biodegradable, thermally degradable, and photolytically degradable polyelectrolytes. Hydrolytically degradable polymers known in the art include for example, certain polyesters, polyanhydrides, polyorthoesters, polyphosphazenes, and polyphosphoesters. Biodegradable polymers known in the art, include, for example, certain polyhydroxyacids, polypropylfumarates, polycaprolactones, polyamides, poly(amino acids), polyacetals, polyethers, biodegradable polycyanoacrylates, biodegradable polyurethanes and polysaccharides. For example, specific biodegradable polymers that may be used in the present invention include but are not limited to polylysine, poly(lactic acid), poly(glycolic acid), poly(caprolactone), poly(lactide-co-glycolide) (PLG), poly(lactide-co-caprolactone) (PLC), and poly(glycolide-co-caprolactone) (PGC). Those skilled in the art will recognize that this is an exemplary, not comprehensive, list of biodegradable polymers. The properties of these and other polymers and methods for preparing them are further described in the art. See, for example, U.S. Pat. Nos. 6,123,727; 5,804,178; 5,770,417; 5,736,372; 5,716,404 to Vacanti; 6,095,148; 5,837,752 to Shastri; 5,902,599 to Anseth; 5,696,175; 5,514,378; 5,512,600 to Mikos; 5,399,665 to Barrera; 5,019,379 to Domb; 5,010,167 to Ron; 4,806,621; 4,638,045 to Kohn; and 4,946,929 to d'Amore. The contents of these references are fully incorporated by reference herein. Of course, co-polymers, mixtures, and adducts of these polymers may also be employed.

The anionic polyelectrolytes may be degradable polymers with anionic groups distributed along the polymer backbone. The anionic groups, which may include carboxylate, sulfonate, sulphate, phosphate, nitrate, or other negatively charged or ionizable groupings, may be disposed upon groups pendant from the backbone or may be incorporated in the backbone itself. The cationic polyelectrolytes may be degradable polymers with cationic groups distributed along the polymer backbone. The cationic groups, which may include protonated amine, quaternary ammonium or phosphonium-derived functions or other positively charged or ionizable groups, may be disposed in side groups pendant from the backbone, may be attached to the backbone directly, or can be incorporated in the backbone itself.

For example, a range of hydrolytically degradable amine containing polyesters bearing cationic side chains have been developed. Examples of these polyesters include poly(L-lactide-co-L-lysine), poly(serine ester), poly(4-hydroxy-L-proline ester), and poly[α-(4-aminobutyl)-L-glycolic acid].

In addition, poly(β-amino ester)s, prepared from the conjugate addition of primary or secondary amines to diacrylates, are suitable for use with the invention. Alternatively, a co-polymer may be used in which one of the components is a poly(β-amino ester). Poly(β-amino ester)s are described in U.S. Pat. No. 6,998,115, the contents of which are fully incorporated by reference herein.

Alternatively or additionally, zwitterionic polyelectrolytes may be used. Such polyelectrolytes may have both anionic and cationic groups incorporated into the backbone or covalently attached to the backbone as part of a pendant group. Such polymers may be neutrally charged at one pH, positively charged at another pH, and negatively charged at a third pH. For example, a film may be deposited by LBL deposition using dip coating in solutions of a first pH at which one layer is anionic and a second layer is cationic. If the film is put into a solution having a second different pH, then the first layer may be rendered cationic while the second layer is rendered anionic, thereby changing the charges on those layers.

The composition of the polyanionic and polycationic layers can be fine-tuned to adjust the degradation rate of each layer within the film. For example, the degradation rate of hydrolytically degradable polyelectrolyte layers can be decreased by associating hydrophobic polymers such as hydrocarbons and lipids with one or more of the layers. Alternatively, the polyelectrolyte layers may be rendered more hydrophilic to increase their hydrolytic degradation rate. In certain embodiments, the degradation rate of a given layer can be adjusted by including a mixture of polyelectrolytes that degrade at different rates or under different conditions. In other embodiments, the polyanionic and/or polycationic layers may include a mixture of degradable and non-degradable polyelectrolytes. Any non-degradable polyelectrolyte can be used with the present invention. Exemplary non-degradable polyelectrolytes that could be used in thin films are include poly(styrene sulfonate) (SPS), poly(acrylic acid) (PAA), linear poly(ethylene imine) (LPEI), poly(diallyldimethyl ammonium chloride) (PDAC), and poly(allylamine hydrochloride) (PAH).

Alternatively or additionally, the degradation rate may be fine-tuned by associating or mixing non-biodegradable, yet biocompatible polymers (polyionic or non-polyionic) with one or more of the polyanionic and/or polycationic layers. Suitable non-biodegradable, yet biocompatible polymers are well known in the art and include polystyrenes, certain polyesters, non-biodegradable polyurethanes, polyureas, poly(ethylene vinyl acetate), polypropylene, polymethacrylate, polyethylene, polycarbonates, and poly(ethylene oxide)s.

### EXEMPLIFICATION

### Example 1

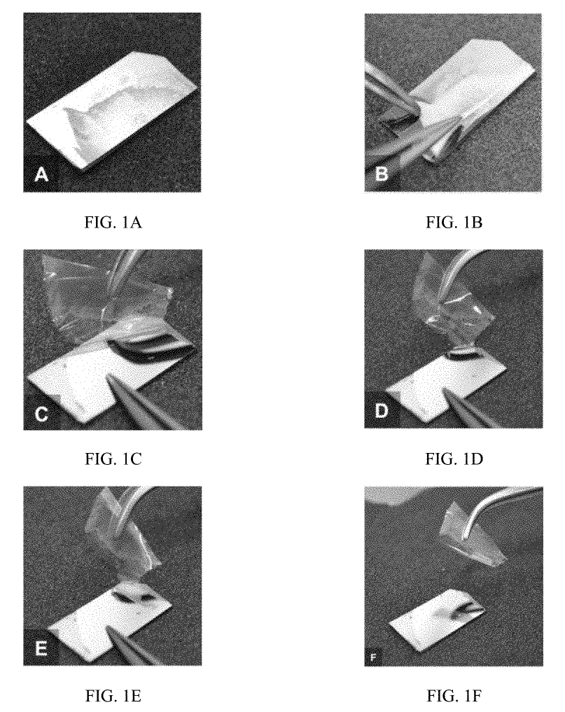

The present disclosure provides bilayer films of (Polymer 2/heparin)₂₄₀ assembled using the spray assisted layer-by-layer (LbL) technique that are peelable into free-standing films (FIG. 1). These films are composed of solely biodegradable materials of Polymer 2 (Poly 2) and heparin with adhesive and controlled drug release properties. The chemical structure of Poly 2 is represented below:

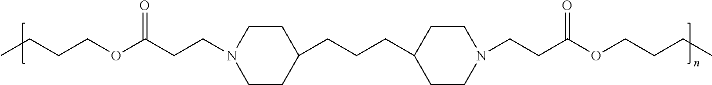

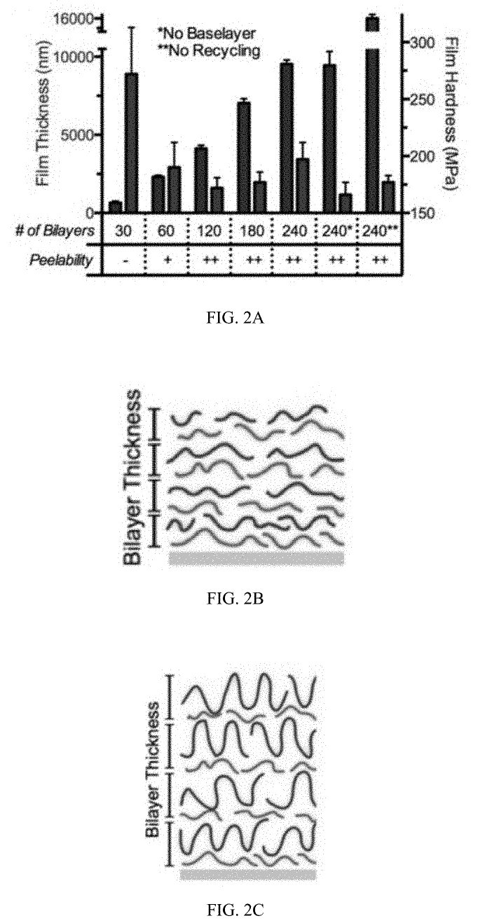

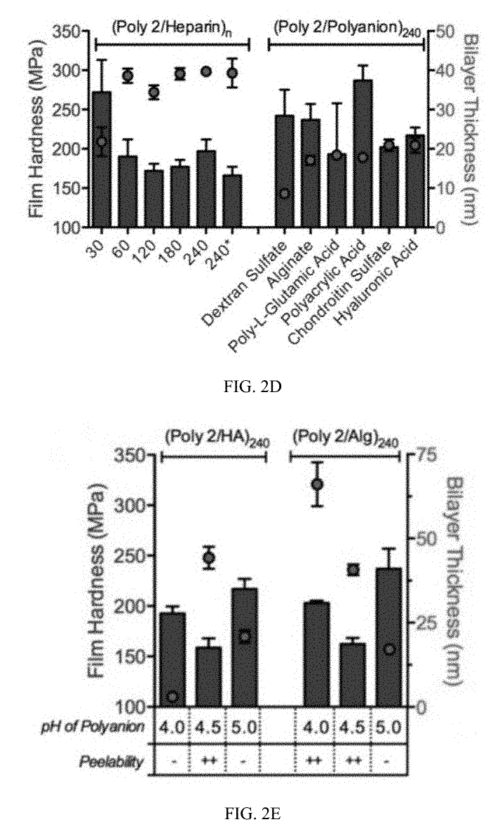

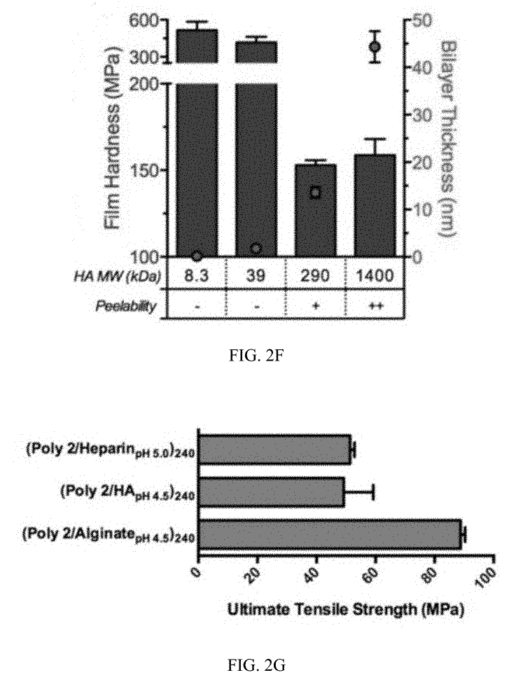

To further investigate this potentially streamlined approach to a free-standing and adhesive controlled drug release film, the mechanical properties of this (Poly 2/heparin)ₙ film were examined as a function of layers (i.e., film thickness). It was discovered that in (Poly 2/heparin)ₙ films, 60-bilayer (or ˜2.5 μm) films could be peeled to some degree but lacked the integrity to be completely removed in a single continuous piece. Thicker films (≧120 bilayers) could be easily peeled whereas a thinner film (30 bilayers) could not be separated from the substrate (FIG. 2A). Non-peelable films were harder as measured by nanoindentation, while softer films were easily peelable (FIG. 2A). Certain parameters were not responsible for this effect, namely the use of a recycling spray-LbL system or baselayer of (polyethylenimine/polysulfonate)₁₀.

Past studies of LbL films have shown that deposition of polyelectrolytes with conformational flexibility resulted in relatively softer films whereas those composed of conformationally rigid polyelectrolytes were harder. Additionally, there was no observed relationship with elastic modulus. The present disclosure similarly shows these properties (Table 1). Conformational flexibility has also been found as an important property for free-standing films composed of a polycation and the mineral clay montmorillonite; loopier and less charged polyelectrolytes are more ductile than films of highly charged polyelectrolytes because of increased entanglements and flexibility. Thus, it was hypothesized that the key property in the films of the present disclosure is the polymer's conformational flexibility where films composed of extended and rigid polyelectrolytes (FIG. 2B) are non-peelable while those of conformationally flexible polyelectrolytes (FIG. 2C) are peelable. One characteristic property of the conformational flexibility of polyelectrolytes deposited in LbL films is the bilayer thickness and so when examining this and hardness of (Poly 2/heparin)ₙ films with regards to bilayers deposited (FIG. 2D), it was discovered that there is a marked increase in bilayer thickness at 60 bilayers, the same point at which films became peelable. At lower bilayer numbers, the films are subject to substrate-related effects that are nullified once films reach a certain thickness, and are likely the cause of the effects observed at 30-bilayers.

TABLE 1

| | Film | Elastic | | |
|---|---|---|---|---|
| (Poly 2/ | Thickness | Modulus | Hardness | |
| Heparin)ₙ | (μm) | (Gpa) | (MPa) | Peelability* |
| 30 | 0.65 ± 0.11 | 5.78 ± 0.98 | 272 ± 41 | − |
| 60 | 2.31 ± 0.11 | 4.98 ± 0.72 | 190 ± 22 | + |
| 120 | 4.12 ± 0.21 | 4.13 ± 0.23 | 172 ± 9 | ++ |
| 180 | 7.03 ± 0.28 | 4.03 ± 0.22 | 177 ± 9 | ++ |
| 240 | 9.53 ± 0.27 | 5.47 ± 0.41 | 197 ± 15 | ++ |
| 240 (no base layer) | 9.44 ± 0.90 | 4.91 ± 0.49 | 166 ± 11 | ++ |
| 240 (no recycling) | 16.08 ± 1.63 | 3.84 ± 0.09 | 177 ± 6 | ++ |

*Not peelable (−), small patches peelable (+), and complete film easily peelable (++).

To ensure that this was not a property unique to (Poly 2/heparin)ₙ films, assembled analogous (Poly 2/polyanion)₂₄₀ films were assembled under identical conditions and chose polyanions commonly used for LbL assembly: sodium polystyrene sulfonate (SPS), hyaluronic acid (HA), dextran sulfate (DS), alginate (Alg), poly-L-glutamic acid (PGA), polyacrylic acid (PAA), and chondroitin sulfate (CS). These films resulted in a range of thicknesses (˜2-6 μm) and mechanical properties, but were not peelable (Table 2). Comparing the bilayer thickness and hardness of these film architectures to that of (Poly 2/heparin)ₙ (FIG. 2D) suggested that those properties had not been maximized and minimized, respectively. The present disclosure demonstrates that peelability can be conferred to these films, and discusses HA and Alg films in particular due to their biodegradability and established use in therapeutic applications.

TABLE 2
Films assembled in 100 mM sodium acetate, pH 5.0

| | Film | Elastic | Hard- | |
|---|---|---|---|---|
| | Thickness | Modulus | ness | Peel- |
| Film Architecture | (μm) | (Gpa) | (MPa) | ability* |
| (Poly 2/Heparin)₂₄₀ | 9.53 ± 0.27 | 5.47 ± 0.41 | 197 ± 15 | ++ |
| (Poly 2/Hyaluronic Acid)₂₄₀ | 5.01 ± 0.47 | 5.99 ± 0.25 | 217 ± 10 | − |
| (Poly 2/Dextran Sulfate)₂₄₀ | 2.06 ± 0.11 | 4.73 ± 0.64 | 242 ± 33 | − |
| (Poly 2/Alginate)₂₄₀ | 4.10 ± 0.29 | 7.10 ± 0.54 | 237 ± 20 | − |
| (Poly 2/Poly-L-Glutamimc | 4.42 ± 0.17 | 6.17 ± 1.76 | 193 ± 65 | − |
| Acide)₂₄₀ | | | | |
| (Poly 2/Polyacrylic Acid)₂₄₀ | 4.28 ± 0.18 | 8.18 ± 0.46 | 287 ± 19 | − |
| (Poly 2/Chondroitin | 5.02 ± 0.35 | 6.52 ± 0.21 | 202 ± 4 | − |
| Sulfate)₂₄₀ | | | | |

*Not peelable (−), small patches peelable (+), and complete film easily peelable (++).

Since HA (pKa˜2.917) and Alg (pKa˜3.2-3.418) are polyacids, acidification of their solutions from pH 5.0 will reduce their charge density thereby increasing their conformational flexibility in these films. For (Poly 2/HA)₂₄₀ films, lowering the polyanion pH from 5.0 to 4.5 doubled the bilayer thickness while reducing film hardness by a quarter, which ultimately resulted in peelable films (FIG. 2E). Further acidification to pH 4.0 appeared to deteriorate film assembly (˜3 nm per bilayer) and is likely due HA's poor charge density. Treating (Poly 2/Alg)₂₄₀ films in the same manner showed that decreasing the polyanion pH from 5.0 to 4.5 increased bilayer thickness and decreased film hardness resulting in the films becoming peelable. Interestingly, the further acidification of the polyanion pH to 4.0 resulted in even thicker films, but also an increased film hardness, with the same peelability. This may be due to its capability of forming hydrogen bonding crosslinks at low pH. As an additional demonstration to the importance of conformational flexibility and polymeric entanglements, the effect of polymer molecular weight is discussed. With (Poly 2/HA_{pH 4.5})₂₄₀ films, it is apparent that with decreasing HA molecular weight, the bilayer thickness correspondingly decreases (FIG. 2F and Table 3), which can be expected with the weaker multivalent crosslinks provided by the polyanion. Furthermore, for molecular weights of 33 kDa and 8.3 kDa, the dramatically increased film hardness suggests substrate related effects associated with the extremely thin films.

TABLE 3
Mechanical properties of (Poly2/HA_{pH 4.5})₂₄₀ films as a
function of HA molecular weight

| (Poly 2/ | | | | |
|---|---|---|---|---|
| HA_{pH 4.5})₂₄₀ | Film | Elastic | | |
| Films | Thickness | Modulus | Hardness | Peel- |
| M_{w} of HA | (μm) | (GPa) | (MPa) | ability |
| 8.3 kDa | 0.038 ± 0.002 | 11.85 ± 1.13 | 517 ± 68 | − |
| 39 kDa | 0.429 ± 0.046 | 9.21 ± 0.84 | 416 ± 45 | − |
| 290 kDa | 3.26 ± 0.28 | 4.07 ± 0.11 | 153 ± 3 | + |
| 1400 kDa | 10.64 ± 0.80 | 4.29 ± 0.29 | 158 ± 9 | ++ |

*Not peelable (−), small patches peelable (+), and complete film easily peelable (++)

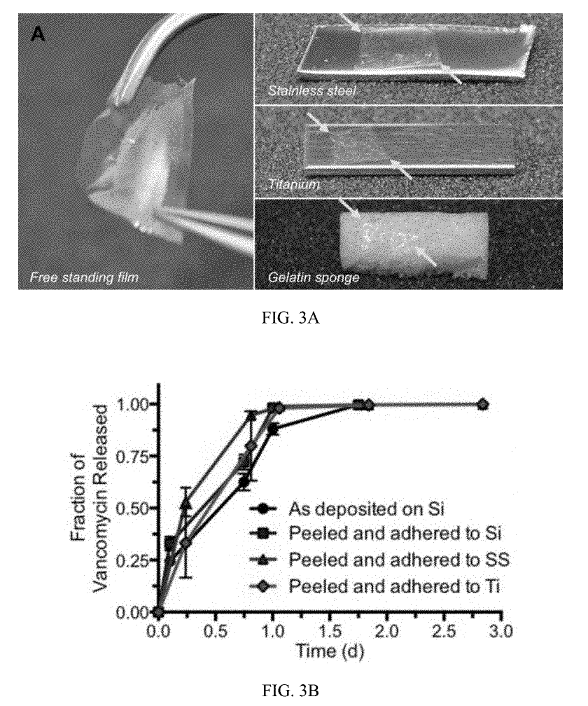

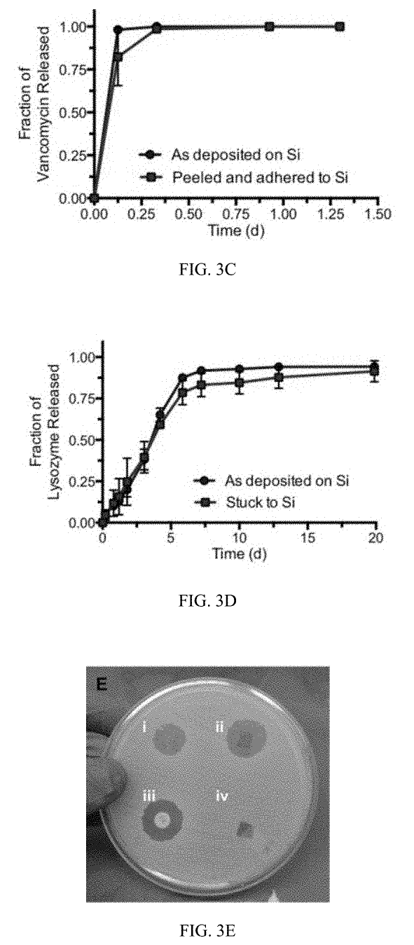

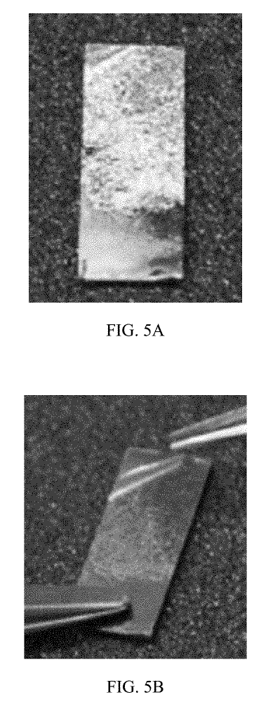

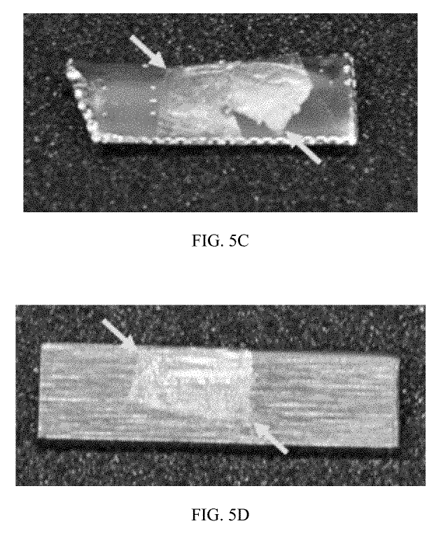

For the films that were readily peelable, their mechanical integrity was examined by measuring their ultimate tensile strength (UTS). Both (Poly 2/heparin_{pH 5.0})₂₄₀ and (Poly 2/HA_{pH 4.5})₂₄₀ films showed similar UTS values whereas (Poly 2/Alg_{pH 4.5})₂₄₀ showed significantly higher strength (FIG. 2G), which may be due to the additional hydrogen bonding capability. The measured UTS values were within the range of previously described free standing LbL polymer films. We next aimed to demonstrate the application of these peelable free-standing films as controlled drug release “stickers” that are capable of re-adhering to new substrates without requiring adhesives, which are often non-degradable and possibly toxic. The film assembly is initiated by deposition of a polycation to the negatively charged silicon surface and so upon peeling, the newly exposed surface of the (Poly 2/heparin/lysozyme/heparin)₂₄₀ films are positively charged. These peeled films can be re-adhered to silicon, stainless steel, titanium, and gelatin sponges (FIG. 3A) without substrate pretreatment. This process was similarly completed with other films, such as (Poly 2/heparin/vancomycin/heparin)₂₄₀ (FIG. 5). To ensure an intimate interaction between the film and new surface, films were briefly hydrated with humidified air, which allowed them to flex and mold to the surface.

To confirm that the process of peeling and re-adherence to new substrates did not affect the controlled release properties of the films, the vancomycin release from (Poly 2/heparin/vancomycin/heparin)₂₄₀ films into the physiologically relevant solution of phosphate buffered saline, pH 7.4 at 37° C., was examined. As shown in FIG. 3B, the release profile is similar between films as deposited on Si to those peeled and re-adhered to new substrates of Si, SS, and Ti. In fact, the peeling and re-adherence process did not significantly affect the release properties of the other film architectures studied, including vancomycin from (Poly 2/Alg/vancomycin/Alg)₂₄₀ films (FIG. 3C) and lysozyme from (Poly 2/heparin/lysozyme/heparin)₂₄₀ films (FIG. 3D). Furthermore, the total drug loadings of films as deposited and re-adhered to the new substrates were the same for vancomycin (Table 4) and lysozyme loaded films (Table 5). Titration of the vancomycin activities upon elution from (Poly 2/heparin/vancomycin/heparin)₂₄₀ and (Poly 2/Alg/vancomycin/Alg)₂₄₀ films shows that the activity is unaffected as determined by the minimum inhibitory concentration (Table 4) and the retained activity of the latter film can be similarly visualized by a Kirby Bauer assay (FIG. 3E).

TABLE 4
Minimum inhibitory concentrations (MIC) of vancomycin
released from films as deposited or re-adhered

| | | |
|---|---|---|
| | Vancomycin loading | MIC |
| Film | in films (μg/cm²) | (μg/mL) |

(Poly 2/Heparin_{pH 5.0}/Vancomycin/Heparin_{pH 5.0})₂₄₀

| | | |
|---|---|---|
| As deposited to Si | 55.7 ± 1.5 | 1.00 ± 0.08 |
| Re-adhered to Si | 54.9 ± 0.8 | 1.00 ± 0.15 |
| Re-adhered to SS | 58.6 ± 1.3 | 1.00 ± 0.31 |
| Re-adhered to Ti | 59.6 ± 2.2 | 1.00 ± 0.06 |

(Poly 2/Alginate_{pH 4.5}/Vancomycin/Alginate_{pH 4.5})₂₄₀

| | | |
|---|---|---|
| As deposited to Si | 24.3 ± 3.9 | 1.00 ± 0.15 |
| Re-adhered to Si | 23.5 ± 5.1 | 1.00 ± 0.02 |

TABLE 5
Lysozyme loading in (Poly
2/Heparin/Lysozyme/Heparin)₂₄₀ films

| | Vancomycin loading |
|---|---|
| Film | (μg/cm²) |
| As deposited to Si | 78.9 ± 0.8 |
| Re-adhered to SS | 80.7 ± 4.8 |

Due to the facile nature of coating surfaces with these controlled drug release stickers, we could rapidly deposit multiple stickers, potentiating multi drug films. To demonstrate this, we assembled three (Poly 2/heparin/Lys/heparin)₂₄₀ films, each containing a different fluorescently tagged lysozyme to enable their visualization.

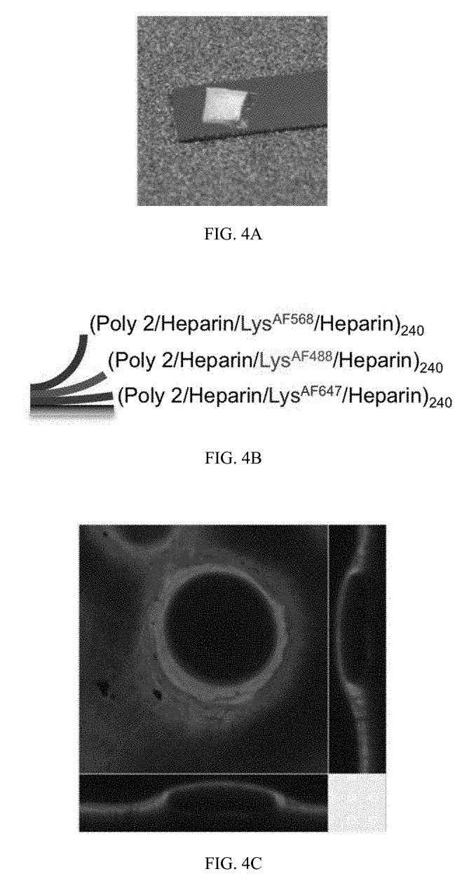

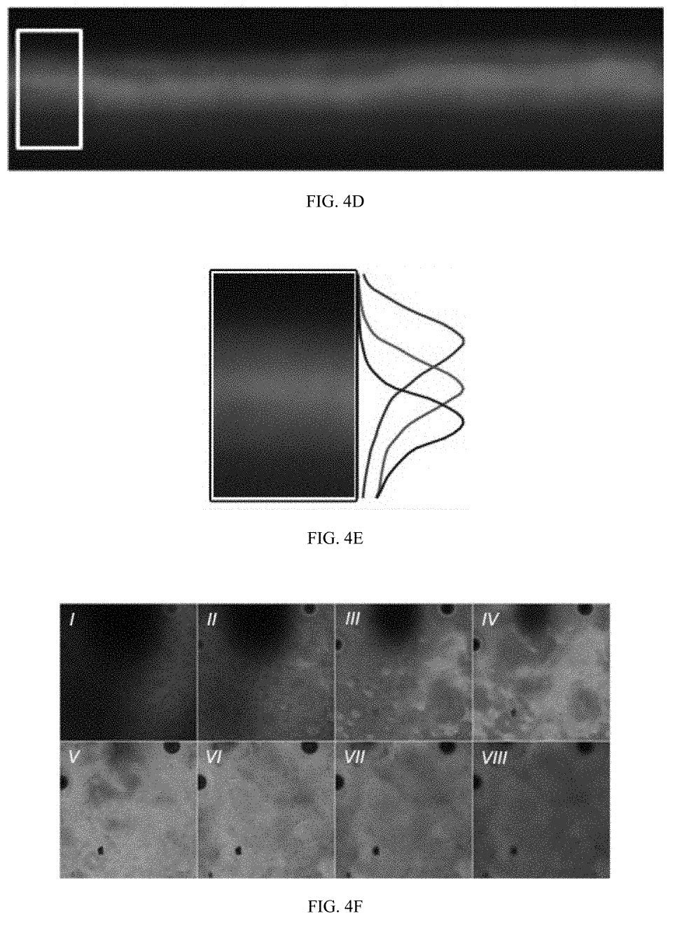

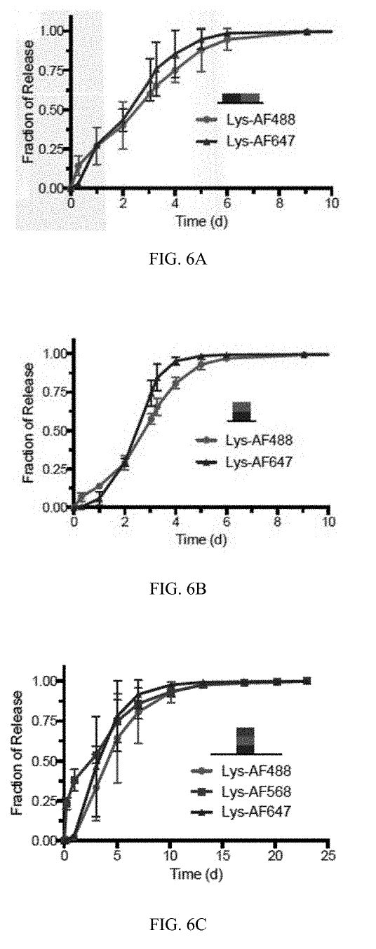

We cut pieces of each film and first re-adhered the film containing Lys^{AF647} (blue) with humidified air, then after a brief moment to allow for the film to dry, repeated the process with the Lys^{AF488} film (green) and finally the Lys^{AF568} film (red). The final multi-film composite is shown in FIG. 4A and schematically shown in FIG. 4B. Generally, the films were deposited smoothly without defects and we found we that in some instances we were able to trap bubbles under or between the films (FIG. 4C). Examination of this multi-film composite by confocal microscopy shows the spatial sequestration of each fluorescently labeled lysozyme to their respective films with minimal, if any, diffusion between films (FIGS. 4D and E). Montage of the z-stack slices beginning at the top and progressing at 3.1 μm intervals through the film (FIG. 4F, from i to viii) show this progression through the layers. For these films, as well as other potential multi-film configurations (i.e., stacked, adjacent, partially stacked, etc.) it is important that the release kinetics of the loaded drugs are not significantly affected. Following the release of fluorescently labeled lysozyme in two films peeled and then re-adhered next to each other on a silicon substrate did not show an impactful difference to the same films in a stacked configuration (FIGS. 6A and 6B, respectively). Furthermore, it did not appear impactful whether one film was buried underneath another, as the release profiles from the upper and lower films were similar (FIG. 6B). This lack of effect was also observed for three films in a stacked configuration (FIG. 6C). The lack of effect suggests that the controlled release is due to the intimate interactions between lysozyme and the polyelectrolyte matrix during film assembly and are not affected by the relatively macroscopic effects of stacking of films.

Peelable free-standing controlled drug release films that are capable of adhesion to biomedically relevant materials opens a potential controlled drug delivery avenue that was previously unavailable because of materials and engineering limitations. The present results indicate that it is possible to generate such coatings using solely biodegradable materials in an all-aqueous assembly process, thus eliminating the potentially harsh conditions or toxic solvents necessary in other methods that may denature or degrade the loaded drugs or cause toxicity. We believe the utilization of pH to confer peelability to these films is a facile yet powerful approach that could potentially be applied to the multitude of functional films already described.

### Example 2

Sodium polystyrene sulfonate (SPS, M_{W}=70 kDa), poly-L-glutamic acid (PGA, MW=50-100 kDa), and sodium alginate (Alg, M_{W}=120-190 kDa) were obtained from Sigma Aldrich, and linear poly(ethylenimine) (LPEI, M_{W}=50 kDa) and polyacrylic acid (PAA, M_{W}=50 kDa) from Polysciences, and heparin sodium salt from Celsius Laboratories. Dextran sulfate sodium salt (500 kDa) was from Calbiochem. Hyaluronic acid (HA, M_{W}=8.3, 39, 290, and 1400 kDa) was obtained from Lifecore Biomedical. Cation adjusted Mueller Hinton broth (CaMHB) and agar were obtained from BD. Polymer 2 (Poly 2) was synthesized as previously described. All other materials were obtained from Sigma Aldrich, unless noted otherwise. All materials were used without further purification.

Film Construction

Silicon slides (Silicon Quest Int'l) of 2.5 cm×2.5 cm were prepared by cleaning with methanol and water, then drying with nitrogen gas, and plasma etching for at least 1 min. To assemble the bilayer and tetralayer films, baselayered slides of (LPEI/SPS)₁₀ were mounted onto a previously described automatic recycling spray system. Tetralayer films containing lysozyme were assembled using the following spray times for each of the polyanions used: 1 sec of 2 mg/mL Poly 2 solution, 3 sec of water, 1 sec of 2 mg/mL polyanion solution, 3 sec of water, 1 sec of 0.5 mg/mL lysozyme solution, 3 sec of water, 1 sec of 2 mg/mL polyanion solution, and 3 sec of water. A wait time of 5 sec was used between each step of the sprayer sequence. This cycle (constituting one tetralayer) was repeated to make 240 tetralayer films. The tetralayer films containing vancomycin followed the same procedure, but used 2 mg/mL vancomycin solution in place of the lysozyme solution. The bilayer films were assembled using a similar procedure with the following spray times: 1 sec of 2 mg/mL Poly 2 solution, 3 sec of water, 1 sec of 2 mg/mL polyanion solution, and 3 sec of water. A 5 sec wait time was also used between each step of the bilayer sprayer sequence. All polymer/protein solutions were formulated in 100 mM sodium acetate buffer, pH 5.0. Aerosolization of solutions was performed with airbrushes (Badger 200NH) with 15 psi and 0.05 mL/sec for each of the polymer/protein solutions. The water wash flow rate varied from 0.05 mL/sec to 0.1 mL/sec depending on the protein used in the tetralayer architecture, vancomycin or lysozyme, respectively. For all bilayer films the water flow rate remained at 0.1 mL/sec. Solution volumes remained constant at 6 mL for all assembled films.

Film Characterization

Assembled films were analyzed for their peelability, i.e., how easily the film was separated from the substrate. The films and silicon substrate were cut using a diamond-tipped pen to expose a corner of the film from the center of the substrate. Tweezers were used to peel the edge of the film away from the substrate. The ease of this process was separated into the following categories: peeled with ease, peeled with difficulty and ripped easily, only small strips could be peeled from substrate, and could not peel.

Films were peeled from their silicon substrate and re-adhered to a different silicon wafer, polished titanium, or polished stainless steel and the release characteristics from the chosen substrate were compared between the peeled and unpeeled films. To improve the level of bonding to the new substrate, peeled films were misted with water from a humidifier for 5 sec to hydrate the film. For release characterization, film samples, both peeled and unpeeled, were incubated in 500 μL of phosphate buffered saline (PBS), pH 7.4 (Gibco) at 37° C. and transferred to fresh aliquots at different time intervals. Lysozyme concentration was measured using a Bicinchoninic Acid (BCA) assay kit (Pierce Biotechnology) and described here briefly. A 25 μL sample was mixed with 200 μL of reagent and incubated at 37° C. for 30 min according to the manufacturer's protocol. The absorbance was measured at 562 nm with a microplate reader (Tecan Infinite M200) and compared to a lysozyme calibration curve.

Vancomycin concentration in solution was determined by HPLC and antimicrobial activity was characterized against Staphylococcus aureus (ATCC 25923) in a microdilution assay to determine the minimum inhibitory concentration, as previously described. Antimicrobial susceptibility in a Kirby Bauer assay was conducted by streaking a CaMHB agar plate with an overnight culture of S. aureus in CaMHB diluted to ˜108 cells/mL and placing ˜1×1 cm of (Poly 2/Alg/vancomycin/Alg)₂₄₀ supported on Si (either as-deposited or peeled and re-adhered) face down onto these plates. An uncoated piece of Si was used as a negative control and a susceptibility test disc containing 30 μg of vancomycin (BD BBL Sensi-Disc) was used as a positive control.

Thickness of assembled films was analyzed by profilometry (Dektak 150 Profilometer) with a 2.5 μm stylus tracked across razor-scored films. Film hardness and elastic moduli were measured by nanoindentation of 0.5 cm×0.5 cm sections of each film were used to test the mechanical properties of each film.

Ultimate tensile strength of peeled, free-standing films were measured with a dynamic mechanical analyzer (DMA Q800, TA Instruments). Films were equilibrated at 30° C. for 5 min prior to measuring the stress during displacement at 1 N/min.

All publications and patents mentioned herein are hereby incorporated by reference in their entirety as if each individual publication or patent was specifically and individually indicated to be incorporated by reference. In case of conflict, the present application, including any definitions herein, will control.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A multilayer film, comprising:
a plurality of repeating multilayer units,
wherein each multilayer unit comprises at least one conformationally flexible layer comprising a conformationally flexible polyelectrolyte.

2. The multilayer film of claim 1, wherein the conformationally flexible polyelectrolyte is a conformationally flexible polyanion.

3. The multilayer film of claim 1, wherein each multilayer unit further comprises at least one layer comprising a polycation.

4. The multilayer film of claim 1, wherein the multilayer film comprises at least 15 multilayer units.

5. The multilayer film of claim 4, wherein the multilayer film comprises at least 30 multilayer units.

6. The multilayer film of claim 1, wherein a pH of each conformationally flexible layer is less than or equal to 5.0.

7. The multilayer film of claim 1, wherein the multilayer film is self-supporting.

8. The multilayer film of claim 3, wherein the polycation is selected from a polyester, a polyanhydride, a polyorthoester, a polyphosphazene, and a polyphosphoester.

9. The multilayer film of claim 8, wherein the polycation is a polyester.

10. The multilayer film of claim 9, wherein the polycation is selected from poly(L-lactide-co-L-lysine), poly(serine ester), poly(4-hydroxy-L-proline ester), and poly[α-(4-aminobutyl)-L-glycolic acid].

11. The multilayer film of claim 9, wherein the polycation is a poly(β-amino ester).

12. The multilayer film of claim 11, wherein the poly(β-amino ester) is a polymer having a repeat unit represented by the following structural formula:

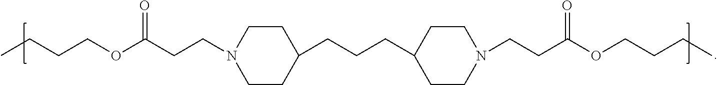

13. The multilayer film of claim 1, wherein each conformationally flexible layer comprises a polymer selected from sodium polystyrene sulfonate, hyaluronic acid, dextran sulfate, alginate, poly-L-glutamic acid, polyacrylic acid, and chondroitin sulfate.

14. The multilayer film of claim 1, wherein each conformationally flexible layer comprises hyaluronic acid and has a pH of 4.5.

15. The multilayer film of claim 1, wherein each conformationally flexible layer comprises alignate and has a pH of 4.5.

16. The multilayer film of claim 1, wherein each conformationally flexible layer comprises alignate and has a pH of 4.0.

17. The multilayer film of claim 1, wherein each multi-layer unit comprises a therapeutic agent.

18. The multilayer film of claim 1, wherein an average thickness of the repeating multilayer units is at least 25 nm.

19. A method of assembling a multilayer film, comprising:
providing a substrate;
applying to the substrate a solution of a conformationally flexible polycation, wherein the solution of the conformationally flexible polycation has a pH of less than or equal to 5.0, thereby depositing a conformationally flexible layer; and
applying a solution of a polyanion to the conformationally flexible layer, thereby depositing a polyanion layer

20. The method of claim 19, further comprising:
applying to the polyanion layer the solution of the conformationally flexible polycation, wherein the solution of a conformationally flexible polycation has a pH of less than or equal to 5.0, thereby depositing at least a second conformationally flexible layer; and
applying the solution of the polyanion to the second conformationally flexible layer, thereby depositing at least a second polyanion layer, thereby depositing a plurality of multilayer units on the substrate; and
removing the plurality of multilayer units from the substrate.
